(19) United States
(12) Patent Application Publication
(10) Pub. No.: US 2003/0118532 A1
(43) Pub. Date: Jun. 26, 2003

(54) Water-in-oil emulsified make-up composition

(75) Inventors: Toshiya Taniguchi, Kobe (JP); Kojo Tanaka, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/300,250

(22) Filed: Nov. 20, 2002

Related U.S. Application Data
(63) Continuation of application No. PCT/US00/15164, filed on Jun. 1, 2000.

(51) Int. Cl. A61K 7/021

(52) U.S. Cl. 424/63

(57) ABSTRACT

Disclosed is a water-in-oil emulsified make-up composition comprising by weight: from about 25% to about 40% of a volatile silicone oil; from about 0.5% to about 8% of a non-volatile oil; from about 1% to about 5% of a solid wax; from about 1% to about 5% of a lipophilic surfactant having an HLB of less than about 8; from about 25% to about 35% of hydrophobically treated pigments; and water in an amount such that the total of the volatile silicone oil and water is at least about 50%.

### CROSS REFERENCE TO RELATED APPLICATIONS

This application is a continuation of International Application No. PCT/US/00/15164, filed Jun. 1, 2000.

### FIELD OF THE INVENTION

The present invention relates to make-up compositions which have a water-in-oil emulsion phase type. Specifically, the present invention relates to make-up compositions which have specified levels of components which provide the benefits of both a solid foundation and a liquid foundation. The present invention also relates to make-up compositions containing water-soluble skin treatment agents such as niacinamide.

### BACKGROUND

A foundation composition can be applied to the face and other parts of the body to even skin tone and texture and to hide pores imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders or anhydrous oil and wax compositions. Emulsion-type foundations in the form of liquid are suitable in that they provide moisturizing effects by the water and water-soluble skin treatment agents incorporated. These liquid form foundations, however, are less convenient to use and carry for the consumer. On the other hand, solid foundations packaged in compacts are suitable for use by the consumer, however, are typically less efficient than liquid form foundations in terms of moisturizing the skin and coverage of the skin.

Foundation compositions in the form of solid, yet water-in-oil emulsion have been suggested. Such emulsion solid foundations can been filled in a wide variety of packaging, including compacts, and is increasing popularity among Asian consumers. References which disclose such foundation compositions are JPA 2-88511, 3-261707, and U.S. Pat. No. 5,362,482. While such emulsion solid foundations have been successful in meeting the drawbacks of conventional liquid form foundations and solid foundations to a certain degree, further improvement is desired. For example, it is known that by increasing the level of water and components intended to moisturize the skin, the fresh and light feel to the skin may be deteriorated. In another example, it is known that, by increasing components which provide smoothness and good spreadability upon application, the appearance on the skin may become powdery. Further, while addressing the desired improvements, the foundation product must be stable during a typical shelf-life of these foundations, and capable of being manufactured at a reasonable cost.

Based on the foregoing, there is a need for a make-up composition which is solid in ambient temperature, provide improved moisturizing benefit to the skin, yet also provide good spreadability to the skin and leave the skin with a fresh and light feel.

### SUMMARY

The present invention is directed to a water-in-oil emulsified make-up composition comprising by weight:

(a) from about 25% to about 40% of a volatile silicone oil;

(b) from about 0.5% to about 8% of a non-volatile oil;

(c) from about 1% to about 5% of a solid wax;

(d) from about 1% to about 5% of a lipophilic surfactant having an HLB of less than about 8;

(e) from about 25% to about 35% of hydrophobically treated pigments; and

(f) water in an amount such that the total of the volatile silicone oil and water is at least about 50%;

which satisfies the need for a make-up composition which is solid in ambient temperature, provide improved moisturizing benefit to the skin, yet also provide good spreadability to the skin and leave the skin with a fresh and light feel.

These and other features, aspects, and advantages of the present invention will become evident to those skilled in the art from a reading of the present disclosure with the appended claims.

### DETAILED DESCRIPTION

The following is a list of definitions for terms used herein.

“Comprising” means that other steps and other ingredients which do not affect the end result can be added. This term encompasses the terms “consisting of” and “consisting essentially of”.

All percentages are by weight of total composition unless specifically stated otherwise.

All cited references are incorporated herein by reference in their entireties. Citation of any reference is not an admission regarding any determination as to its availability as prior art to the claimed invention.

All ratios are weight ratios unless specifically stated otherwise.

The present invention, in its product and process aspects, is described in detail as follows.

Volatile Silicone Oil

The composition of the present invention comprises a volatile silicone oil by weight of the entire composition at from about 25% to about 40%, preferably 30% to about 40%. The amount of the volatile silicone oil is controlled so that the total of the volatile silicone oil and water is more than about 50% of the entire composition. Without being bound by theory, the species and levels of the volatile silicone oil herein is believed to provide improved refreshing and light feeling to the skin, without necessarily leaving a dried feeling to the skin.

The volatile silicone oil useful herein are selected from those having a boiling point of from about 60 to about 260° C., preferably those having from 2 to 7 silicon atoms.

The volatile silicone oils useful herein include polyalkyl or polyaryl siloxanes with the following structure (1):

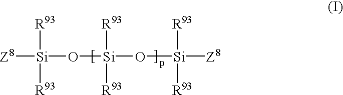

wherein R⁹³ is independently alkyl or aryl, and p is an integer from about 0 to about 5. Z⁸ represents groups which block the ends of the silicone chains. Preferably, R⁹³ groups include methyl, ethyl, propyl, phenyl, methylphenyl and phenylmethyl, Z⁸ groups include hydroxy, methyl, methoxy, ethoxy, propoxy, and aryloxy. More preferably, R⁹³ groups and Z⁸ groups are methyl groups. The preferred volatile silicone compounds are hexamethyldisiloxane, octamethyltrisiloxane, decamethyltetrasiloxane, hexadecamethylheptasiloxane. Commercially available volatile silicone compounds useful herein include octamethyltrisiloxane with tradename SH200C-1cs, decamethyltetrasiloxane with tradename SH200C-1.5cs, hexadecamethylheptasiloxane with tradename SH200C-2cs, all available from Dow Corning.

The volatile silicone oils useful herein also include a cyclic silicone compound having the formula:

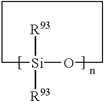

wherein R⁹³ is independently alkyl or aryl, and n is an integer of from 3 to 7.

Preferably, R⁹³ groups include methyl, ethyl, propyl, phenyl, methylphenyl and phenylmethyl. More preferably, R⁹³ groups are methyl groups. The preferred volatile silicone compounds are octamethylcyclotetrasiloxane, decamethylcyclopentasiloxane, tetradecamethylcyclohexasiloxane. Commercially available volatile silicone compounds useful herein include octamethylcyclotetrasiloxane with tradename SH244, decamethylcyclopentasiloxane with tradename DC245 and SH245, and dodeamethylcyclohexasiloxane with tradename DC246; all available from Dow Corning.

Non-Volatile Oil

The composition of the present invention comprises a non-volatile oil by weight of the entire composition at from about 0.5% to about 8%, preferably 2% to about 5%. The amount of the non-volatile oil is controlled so that the ratio of 1) the total of the non-volatile oil, the solid wax, and the lipophilic surfactant; and 2) the hydrophobically treated pigments is from about 1:7 to about 1:1.5, preferably from about 1:5 to about 1:2, more preferably from about 1:5 to about 1:2.5. Without being bound by theory, the species and levels of the non-volatile oil herein is believed to provide improved smoothness to the skin, and also alleviate the powdery look often found when pigments are included at a high level.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil., rice bran oil, china paulownia oil., Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile oils useful herein also include polyalkyl or polyaryl siloxanes with the following structure (I)

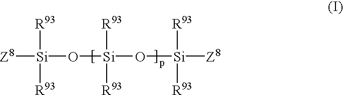

wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Non-volatile oils particularly useful herein are those which have relatively low viscosity. Such low viscosity non-volatile oils are believed to enhance the fresh and light feel when the composition is applied to the skin.

Solid Wax

The composition of the present invention comprises a solid wax by weight of the entire composition at from about 1% to about 5%, preferably 2% to about 4%. The amount of the solid wax is controlled so that the ratio of 1) the total of the non-volatile oil, the solid wax, and the lipophilic surfactant; and 2) the hydrophobically treated pigments is from about 1:7 to about 1:1.5, preferably from about 1:5 to about 1:2, more preferably from about 1:5 to about 1:2.5. Without being bound by theory, the species and levels of the solid wax herein is believed to provide consistency to the composition and coverage to the skin, while not negatively contributing to the spreadability upon application to the skin, and fresh and light feel of the skin.

The solid waxes useful herein are paraffin wax, microcrystalline wax, ozokerite was, ceresin wax, carnauba wax, candellila wax, eicosanyl behenate, and mixtures thereof. A mixture of waxes is preferably used.

Commercially available solid waxes useful herein include: Candelilla wax NC-1630 available from Noda wax, Ozokerite wax SP-1021 available from Strahl & Pitsh, and Eicosanyl behenate available from Cas Chemical.

Lipophilic Surfactant

The composition of the present invention comprises a lipophilic surfactant by weight of the entire composition at from about 1% to about 5%, preferably 1% to about 3%. The lipophilic surfactant herein has an HLB value of less than about 8.

The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in “Surfactant Science Series, Vol. 1: Nonionic Surfactants”, pp 606-13, M. J. Schick (Marcel Dekker Inc., New York, 1966).

The amount of the lipophilic surfactant is controlled so that the ratio of 1) the total of the non-volatile oil, the solid wax, and the lipophilic surfactant; and 2) the hydrophobically treated pigments is from about 1:7 to about 1:1.5, preferably from about 1:5 to about 1:2, more preferably from about 1:5 to about 1:2.5. Without being bound by theory, the species and levels of the lipophilic surfactant herein is believed to provide a stable water-in-oil emulsion in view of the other components of the present invention.

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), as shown below, and mixtures thereof.

(i) dimethicone copolyols having the structure:

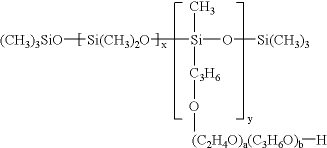

wherein x is an integer from 5 to 100, y is an integer from 1 to 50, a is zero or greater, b is zero or greater, the average sum of a+b being 1-100.

(ii) dimethicone copolyols having the structure:

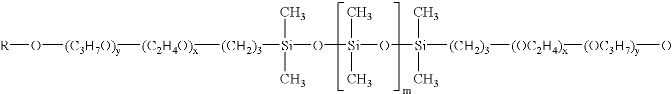

wherein R is selected from the group consisting of hydrogen, methyl, and combinations thereof, m is an integer from 5 to 100, x is independently zero or greater, y is independently zero or greater, the sum of x+y being 1-100.

Commercially available silicone-type surfactants are, for example, DC5225C, BY22-012, BY22-008, SH3746M, SH3771M, SH3772M, SH3773M, SH3775M, SH3748, SH3749, and DC5200, all available from Dow Corning.

In a preferred embodiment, the lipophilic surfactant is a mixture of at least one ester-type surfactant and at least one silicone-type surfactant to provide a stable emulsion for the other essential components of the present invention.

Hydrophobically Treated Pigments

The composition of the present invention comprises a hydrophobically treated pigment by weight of the entire composition at from about 25% to about 35%, preferably from about 25% to about 30%. The amount of the hydrophobically treated pigment is controlled so that the ratio of 1) the total of the non-volatile oil, the solid wax, and the lipophilic surfactant; and 2) the hydrophobically treated pigments is from about 1:7 to about 1:1.5, preferably from about 1:5 to about 1:2, more preferably from about 1:5 to about 1:2.5. Without being bound by theory, the species and levels of the hydrophobically treated pigment herein is believed to provide good wear performance, and is stable in the composition as being stable in the oily continuous phase.

The hydrophobically treated pigments are selected depending on the desired characteristic of the product, for example, shade, coverage, UV protection benefit, and various skin feel.

The base material useful for the hydrophobically treated pigments herein are clay mineral powders such as talc, mica, sericite, silica, magnesium silicate, synthetic fluorphlogopite, calcium silicate, aluminum silicate, bentonite and montomorilonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titate, ultramarine blue, Prussian blue, chromium oxide, chromium hydroxide, cobalt oxide, cobalt titanate, titanium oxide coated mica; organic powders such as polyester, polyethylene, polystyrene, methyl metharylate resin, cellulose, 12-nylon, 6-nylon, styrene-acrylic acid copolymers, poly proprylene, vinyl chloride polymer, tetrafluoroethylene polymer, boron nitride, fish scale guanine, laked tar color dyes, and laked natural color dyes. Such base material are treated with a hydrophobical treatment agent, including:silicone such as Methicone, Dimethicone and perfluoroalkylsilane; fatty material such as stearic acid; metal soap such as aluminium dimyristate; aluminium hydrogenated tallow glutamate, hydrogenated lecithin, lauroyl lysine, aluminium salt of perfluoroalkyl phosphate, and mixtures thereof.

A certain percentage of spherical pigments can be used. In a preferred embodiment, the hydrophobically treated pigments are also selected depending on the oil absorbing capability of the pigments. In one preferred embodiment, pigments having high oil absorbing capability and those having low oil absorbing capability are combined.

Water

The composition of the present invention comprises water in an amount such that the total of the volatile silicone oil and water is more than about 50% of the entire composition, preferably from about 15% to about 25% by weight of the entire composition.

Without being bound by theory, the species and levels of water herein is believed to provide improved refreshing and light feeling to the skin, without necessarily leaving a dried feeling to the skin. Further, this amount of water allows the inclusion of water-soluble skin treatment agents such as niacinamide.

In the present invention, deionized water is typically used. Water from natural sources including mineral cations can also be used, depending on the desired characteristic of the product.

Skin Treatment Agent

The composition of the present invention may further comprise a skin treatment agent by weight of the entire composition at from about 0.1% to about 10%, preferably from about 1% to about 5%.

Skin treatment agents useful herein are niacinamide, panthenol, and mixtures thereof. Niacinamide is particularly preferred in that, when used in a pharmaceutically effective amount, is capable of reducing or alleviating the intensity of chronical spots. Niacinamide is suitably incorporated in the composition by first dissolving in water. Niacinamide and panthenol are commercially available, for example, by Roche.

Humectant

The composition of the present invention may further comprise a humectant by weight of the entire composition at from about 1% to about 15%, preferably 2% to about 7%.

The humectants herein are selected from the group consisting of polyhydric alcohols, water soluble alkoxylated nonionic polymers, and mixtures thereof.

Polyhydric alcohols useful herein include glycerin, propylene glycol, 1,3-butylene glycol, dipropylene glycol, diglycerin, sodium hyaluronate, and mixtures thereof.

Commercially available humectants herein include:glycerin available from Asahi Denka; propylene glycol with tradename LEXOL PG-865/855 available from Inolex, 1,2-PROPYLENE GLYCOL USP available from BASF; 1,3-butylene glycol available from Daisel Kagaku Kogyo; dipropylene glycol with the same tradename available from BASF; diglycerin with tradename DIGLYCEROL available from Solvay GmbH; sodium hyaluronate with tradenames ACTIMOIST available from Active Organics, AVIAN SODIUM HYALURONATE series available from Intergen, HYALURONIC ACID Na available from Ichimaru Pharcos.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such additional components generally are used individually at levels of no more than about 5% by weight of the composition.

The composition of the present invention may further contain a nonvolatile dispersed silicone usually referred to as silicone gum. The term “silicone gum”, as used herein, means a polyorganosiloxane material having a viscosity at 25° C. of greater than or equal to 1,000,000 mPa·s. Silicone gums are believed to provide wearability improvement such as long-lasting effect. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. Commercially available silicone gums are described in General Electric Silicone Rubber Product Data Sheets as SE 30, SE 33, SE 54 and SE 76.

The composition of the present invention may further contain a silicone resin, which are highly crosslinked polymeric siloxane systems. Silicone resins are believed to enhance spreadability and improve the feel to the skin. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred are crosslinked silicone powders with tradenames Trefil E-505C, Trefil E-506C, and 9506 Powder; suspensions of silicone elastomer powders with tradenames BY29-119 and BY29-122; and silicone compound emulsions with tradenames SH5500, SC5570, and SM 5571; all available from Dow Corning.

Other useful silicone resins are silicone resin powders such as the material given the CTFA designation polymethylsilsequioxane, which is commercially available as Tospearl™ from Toshiba Silicones.

Silicone materials and silicone resins in particular, can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO₀₅; D denotes the difunctional unit (CH₃)₂SiO; T denotes the trifunctional unit (CH₃)SiO_{1.5}; and Q denotes the quadri- or tetra-functional unit SiO2. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000. Commercially available MQ resins are, for example, trimethyl siloxy silicate with tradename BY11-018 available from Dow Corning.

The composition of the present invention may further contain a water-soluble polymer. It is believed that water-soluble polymers provide long-lasting effect. Useful water-soluble polymers include sodium carboxymethyl cellulose, polyvinyl pyrrolidone, polyvinyl alcohol, xanthan gum, agar, pulleran, bentonite, and mixtures thereof. Commercially available water-soluble polymers include the Carbopol series available from B.F. Goodrich Company, and PVP K-30 available from G.A.F. Chemicals.

Other components which can be formulated into the compositions of the present invention are; preservatives such as benzyl alcohol, methyl paraben, propyl paraben, imidazolidinyl area, and EDTA and its salts, perfumes, ultraviolet and infrared screening and absorbing agents, and others.

The Composition

The composition of the present invention may be made by a method well known in the art. In a suitable process, the composition is made by the steps of:

1) heating and dissolving the volatile silicone oil, non-volatile oil, solid wax, lipophilic surfactant, and any other hydrophobic material having a high melting point to about 80-85° C. in a sealed tank, to make a lipophilic mixture;

2) adding the hydrophobically treated pigments into such liphophilic mixture and dispersing with a homogenizer at about 75-80° C.;

3) separate from 1) and 2), heating and dissolving in water, skin treatment agents, humectants, when present, and any other hydrophilic material to about 75-80° C.;

4) adding the product of step 2) to the product of step 3) to effect an emulsification; and

5) cooling the obtained emulsion to a temperature of about 60-80° C.

The obtained composition, which is still fluid at such temperature, is filled in an air-tight container and allowed to cool to room temperature typically using a cooling unit. The obtained composition is solid at ambient temperature, and thus can be poured into such container and left to solidify. The air-tight container is typically in a package form of a compact.

The obtained composition preferably has a melting point of less than about 50° C., when measured by “General Tests, Melting Point Method 2” as stated in “The Japanese Standard of Cosmetic Ingredients”. Without being bound by theory, it is believed that this relatively low melting point of the preferred embodiment composition of the present invention provides the improved spreadability and improved fresh and light feeling to the skin. In another preferred embodiment, the DSC thermogram of the composition of the present invention shows a relatively small enthalpy required for melting the composition. Without being bound by theory, it is believed that such small enthalpy leads to less energy required for the composition to be applied to the skin, thereby, also contributing to the improved spreadability and improved fresh and light feeling to the skin.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Examples 1-5

The following make-up compositions are formed by the process described herein:

| NO. | Component | Ex. 1 | Ex. 2 | Ex. 3 | Ex. 4 | Ex. 5 |
|---|---|---|---|---|---|---|
| 1 | Cyclomethicone *1 | 35.9 | 30.95 | 32.95 | 33.3 | 32.8 |
| 2 | Tridecyl isononanoate *2 | 2.0 | 2.0 | 2.0 | 2.0 | 2.0 |
| 3 | Tocopherol acetate *3 | 0.5 | 0.5 | 0.5 | | 0.5 |
| 4 | Candelilla wax *4 | 0.4 | 0.4 | 0.4 | 0.3 | |
| 5 | Ozokerite wax *5 | 2.2 | 2.8 | 2.8 | 2.8 | 2.0 |
| 6 | Eicosanyl Behenate *6 | | 0.4 | 0.4 | 0.4 | 2.5 |
| 7 | Sorbitan isostearate *7 | 2.0 | 2.0 | 2.0 | 1.0 | 1.0 |
| 8 | Dimethicone copolyol *8 | 0.5 | 1.2 | 1.2 | 1.2 | 1.2 |
| 9 | Crosslinked silicone powder | | | 2.0 | | |
| | *9 | | | | | |
| 10 | Trimethyl siloxy silicate *10 | | | | 1.0 | |
| 11 | Hydrophobically Treated | 28.75 | 30.00 | 28.00 | 30.00 | 25.00 |
| | Pigments *11 | | | | | |
| 12 | Deionized water | 20 | 22 | 20 | 20 | 22 |
| 13 | Niacinamide *12 | 2.0 | 2.0 | 2.0 | 2.0 | 5.0 |
| 14 | Panthenol *13 | 0.25 | 0.25 | 0.25 | | 0.5 |
| 15 | Preservative | 0.5 | 0.5 | 0.5 | 0.5 | 0.5 |
| 16 | 1,3 butylene glycol *14 | 5.0 | 5.0 | | 5.0 | 5.0 |
| 17 | Glycerin *15 | | | 5.0 | | |
| 18 | Polyvinylpyrrolidone *16 | | | | 0.5 | |

Definitions of Components
*1 Cyclomethicone: SH245 available from Dow Corning
*2 Tridecyl isononanoate: Crodamol TN available from Croda
*3 Tocopheryl Acetate: DL-a-Tocopheryl Acetate available from Eisai
*4 Candelilla wax: Candelilla wax NC-1630 available from Noda wax
*5 Ozokerite wax: Ozokerite wax SP-1021 available from Strahl & Pitsh
*6 Eicosanyl Behenate: Eicosanyl Behenate available from Cas Chemical
*7 Sorbitan isostearate: Crill 6 available from Croda
*8 Dimethicone copolyol: DC5225C available from Dow Corning
*9 Crosslinked silicone powder: Torayfil E-506C available from Dow Corning
*10 Trimethylsiloxy silicate: BY11-018 available from Dow Corning
*11 Hydrophobically treated pigments:
*12 Niacinamide: Niacinamide available from Roche
*13 Panthenol: DL-Panthenol available from Roche
*14 1,3 Butylene Glycol: 1,3 Butylene Glycol available from Daisel Kagakukougyou
*15 Glycerin: Glycerine USP available USP available from Asahi Denka
*16 Polyvinylpyrrolidone: PVP K-30 available from GAF Chemicals

| | | *Oil Absorbency | |
|---|---|---|---|
| Component | Level (%) | level (ml/g) | Supplier |
| Methicone Treated Titanium | 0-70 | 24.4 | Miyoshi Kasei |
| Dioxide and Talc | | | |
| Methicone Treated Mica | 15-85 | 58.5 | Miyoshi Kasei |
| Dimethicone and Stearic Acid | 0-25 | 23.0 | Miyoshi Kasei |
| Treated Micro Titanium Dioxide | | | |
| Dimethicone Treated Silica | 5-35 | 130.4 | Miyoshi Kasei |
| Methicone Treated Synthetic Mica | 0-35 | — | Topy Kougyou |
| (synthetic fluorphlogopite) | | | |
| Methicone Treated Iron Oxides | 0-70 | — | Daito Kasei |

*Test Method for Oil Absorbency Level: JIS K5101 No. 21

Method of Preparation

The make-up compositions of Examples 1-5 are prepared as follows: component numbers 1 through 10, as present, are heated to dissolve at 82° C. in a sealed tank, followed by adding component number 11, and the mixture is dispersed at 80° C. using a homogenizer to make a lipophilic mixture. Separately, a mixture of component numbers 12 through 18, as present, are heated to dissolve at 80° C. and added to the lipophilic mixture to effect an emulsification. The obtained emulsion is adjusted to a temperature of 70° C. Finally, the emulsion is filled in an air-tight container and allowed to cool to room temperature using a cooling unit.

These embodiments represented by the previous examples have many advantages. For example, they can provide improved moisturizing benefit to the skin, yet also provide good spreadability to the skin and leave the skin with a fresh and light feel.

What is claimed is:

1. A water-in-oil emulsified make-up composition wherein said composition comprises by weight:
(a) from about 25% to about 40% of a volatile silicone oil;
(b) from about 0.5% to about 8% of a non-volatile oil;
(c) from about 1% to about 5% of a solid wax;
(d) from about 1% to about 5% of a lipophilic surfactant having an HLB of less than about 8;
(e) from about 25% to about 35% of hydrophobically treated pigments; and
(f) water in an amount such that the total of the volatile silicone oil and water is at least about 50%.

2. The water-in-oil emulsified make-up composition according to claim 1 wherein said composition further comprises from about 0.1% to about 10% of a skin treatment agent.

3. The water-in-oil emulsified make-up composition according to claim 2 wherein the skin treatment agent is selected from the group consisting of niacinamide, panthenol, and mixtures thereof.

4. The water-in-oil emulsified make-up composition according to claim 2 wherein the skin treatment agent is niacinamide.

5. The water-in-oil emulsified make-up composition according to claim 1 wherein the composition has a melting point of less than about 50° C.

6. The water-in-oil emulsified make-up composition according to claim 1 wherein the weight ratio of the total amount of the non-volatile oil, the solid wax, and the lipophilic surfactant; to the amount of the hydrophobically treated pigments is from about 1:7 to about 1:1.5.

7. The water-in-oil emulsified make-up composition according to claim 1 wherein the non-volatile oil is selected from the group consisting of tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, and mixtures thereof.

8. The water-in-oil emulsified make-up composition according to claim 1 wherein the lipophilic surfactant is selected from the group consisting of ester-type surfactants, silicone-type surfactants, and mixtures thereof.

9. The water-in-oil emulsified make-up composition according to claim 8 wherein the ester-type surfactant is selected from the group consisting of sorbitan isostearate, sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

10. The water-in-oil emulsified make-up composition according to claim 1 wherein said composition further comprises from about 1% to about 15% of a humectant.

11. The water-in-oil emulsified make-up composition according to claim 1 wherein said composition comprises by weight:
(a) from about 30% to about 40% of the volatile silicone oil;
(b) from about 2% to about 5% of the non-volatile oil;
(c) from about 2% to about 4% of the solid wax;
(d) from about 1% to about 3% of the lipophilic surfactant;
(e) from about 25% to about 30% of hydrophobically treated pigments wherein the weight ratio of: the total amount of the non-volatile oil, the solid wax, and the lipophilic surfactant; to the amount of the hydrophobically treated pigments is 1:5 to about 1:2.5;
(f) from about 15% to about 25% water;
(g) from about 1% to about 5% of the skin treatment agent; and
(h) from about 2% to about 7% of a humectant.
